(19) United States
(12) Patent Application Publication
(10) Pub. No.: US 2007/0233096 A1
(43) Pub. Date: Oct. 4, 2007

(54) Dynamic inter-spinous device

(76) Inventor: Javier Garcia-Bengochea, Jacksonville, FL (US)

Correspondence Address: ROGERS TOWERS, P.A., 1301 RIVERPLACE BOULEVARD, SUITE 1500, JACKSONVILLE, FL, 32207, US

(21) Appl. No.: 11/706,895

(22) Filed: Feb. 13, 2007

Related U.S. Application Data
(60) Provisional application No. 60/772,724, filed on Feb. 13, 2006.

(51) Int. Cl. A61F 2/30

(52) U.S. Cl. 606/61

(57) ABSTRACT

A dynamic inter-spinous device having an inferior member and superior member, wherein the combination of a concave member and a convex member allow the inferior and superior members to articulate in multiple directions and planes.

This application claims the benefit of U.S. Provisional Patent Application Ser. No. 60/772,724, filed Feb. 13, 2006.

### BACKGROUND OF THE INVENTION

This invention relates generally to devices known under various names such as prostheses, spacers, implants, or fixation devices that are positioned between or used to connect in a controlled manner the spinous processes of adjacent vertebrae in the spine. More particularly, the invention relates to such devices that allow relative movement of the adjacent spinous processes.

Historically, the treatment of many spinal disorders has involved fixation or fusion techniques where one or more adjacent vertebrae are immobilized using mechanical constructs, such as plates, rods, screws and the like. A developing trend in the treatment of spinal disorders comprises the integration of non-fusion technologies with traditional fusion techniques. The spectrum of non-fusion technologies ranges from total disc replacement to dynamic stabilization to a category of devices that are termed “non-fusion” in that they eliminate motion in all other planes, but which do not involve fusion per se.

Current motion preserving devices are designed as either disc or facet replacement/augmentation devices. Known devices for “non-fusion” stabilization in the inter-spinous region are static devices that essentially eliminate motion in extension, flexion and rotation. They are spacers placed in the distracted inter-spinous region of the lumbar spine that are anchored to the spinous process above and below using Dacron cords or mechanical fasteners. The spacer produces a segmental kyphosis, i.e., and abnormal curvature of the spine, that stretches the ligmentum flavum to lessen central spinal and lateral recess stenosis, i.e., compression or narrowing, opens the neural foramen to reduce foraminal stenosis and eliminates extension to prevent exacerbation of the aforementioned conditions. An advantage of these devices is that they are placed through small, superficial incisions that are not associated with the operative morbidity of conventional spinal exposures for fusion. Thus, they potentially provide a low risk and simple method for treating common conditions in the short and intermediate term while obviating the lengthy recovery associated with fusion techniques. Some such devices are composed of compressible material.

Examples of known inter-spinous devices are found in U.S. Pat. No. 5,011,484 to Breard, U.S. Pat. No. 5,456,722 to McLeod et al., U.S. Pat. No. 5,496,318 to Howland et al., U.S. Pat. No. 5,609,634 to Voydeville, U.S. Pat. No. 5,645,599 to Samani, U.S. Pat. No. 5,725,582 to Bevan et al. U.S. Pat. No. 6,582,433 to Yun, U.S. Pat. No. 6,626,944 to Taylor, U.S. Pat. No. 6,695,842 to Zucherman et al., U.S. Pat. No. 6,733,534 to Sherman, U.S. Pat. No. 6,761,720 to Senegas, U.S. Pat. No. 6,796,983 to Zucherman et al., and U.S. Pat. No. 6,946,000 to Senegas et al.

It is an object of this invention to provide an inter-spinous device that is dynamic rather than static, such that relative movement of adjacent spinous processes is controlled and not totally precluded. It is a further object to provide such a device that is composed of plural members and that is articulated and allows movement in flexion, extension, rotation, lateral bending and translation in any plane. It is a further object to provide such a device that may be used to create a segmental kyphosis to relieve central spinal and neural foraminal stenosis while preserving some motion. It is a further object to provide such a device that preserves distraction of the intervertebral foramen and ligamentum flavum during movement of the spine, especially during extension.

### SUMMARY OF THE INVENTION

The invention comprises a dynamic inter-spinous device that is positioned between adjacent vertebral spinous processes in order to stabilize the region while allowing for some degree of movement in at least two directions, and preferably in all directions, such that the device articulates in flexion, extension, rotation, lateral bending and translation in any plane. The inter-spinous device comprises a superior member and an inferior member that in combination preserve distraction of the intervertebral foramen and ligamentum flavum while allowing for some movement. The superior surface of the superior member and the inferior surface of the inferior member are each provided with a channel, depression or the like adapted to receive the inferior and superior edges, respectively, of the adjacent spinous processes.

The superior and inferior members are mated in a manner that allows for relative movement in a controlled manner. In the preferred embodiment, the inferior surface of the superior member is provided with a concave member and the superior surface of the inferior member is provided with a corresponding convex member. Preferably, the concave member and convex member are generally hemispherical, a configuration referred to as a ball and socket, although other curvilinear surface configurations may be utilized that are not true portions of a sphere. In an alternative embodiment, the socket member and convex member may be semi-cylindrical, such that relative motion is allowed within a vertical plane but precluded horizontally. In another alternative embodiment, the overall configuration of the device is curved and provided with a tapered leading edge for ease of implantation. The depth of the concave socket member is less than the height of the convex member such that the superior member may rotate and pivot relative to the inferior member. Alternatively, the convex member could be raised, mounted on a post, etc., such that even greater pivoting is allowed. The inferior member may be provided with lateral flange members adapted to fit over the superior surface of the inferior lamina to better stabilize the device.

Anchoring means are provided to secure each of the superior and inferior members to adjoining spinous processes or lamina, and the anchoring means may comprise cords, straps, mechanical fasteners or other suitable means.

### BRIEF DESCRIPTION OF THE DRAWINGS

FIG. 1 is a lateral view of one embodiment of the invention.

FIG. 2 is an end view of the embodiment of FIG. 1.

FIG. 3 is a lateral view showing the invention disposed between the spinous processes.

FIG. 4 is an exploded perspective view of an alternative embodiment of the device, wherein the device may pivot for flexion and tension but is not able to rotate.

FIG. 5 is a top view of an alternative embodiment of the invention disposed between the spinous processes.

FIG. 6 is a side view of the embodiment of FIG. 5 disposed between the spinous processes.

FIG. 7 is an end view of the embodiment of FIG. 5 disposed between the spinous processes.

### DETAILED DESCRIPTION OF THE INVENTION

With reference to the drawings, the invention will now be described in detail with regard for the best mode and the preferred embodiment. In general, the invention is a dynamic, articulating inter-spinous spacer or prosthetic device that allows motion in multiple directions and planes, and preferably in all directions and planes, while preserving distraction of the intervertebral foramen and ligamentum flavum during movement of the spine, especially during extension. As used herein, directional terms such as anterior, posterior, lateral, superior and inferior shall be taken to correspond to the standard reference directions in surgical applications when the dynamic inter-spinous device is implanted between adjacent spinous processes.

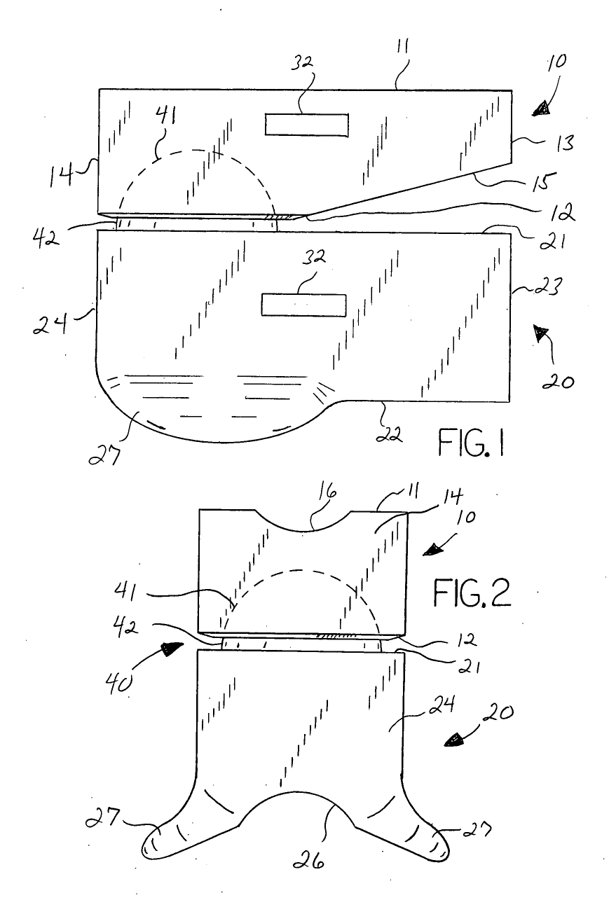

As illustrated in FIGS. 1 and 2, the dynamic inter-spinous device comprises a superior member 10 and an inferior member 20. The superior member 10 comprises a superior surface 11, an inferior surface 12, a posterior portion 13 and an anterior portion 14. Likewise, the inferior member 20 comprises a superior surface 21, an inferior surface 22, a posterior portion 23 and an anterior portion 24. The superior and inferior members 10 and 20 are composed of bio-compatible materials suitable for permanent placement in the human body, and are preferably composed of a relatively rigid material. Alternatively, a material composition that provides for a relatively limited amount of compression and/or flexing may be utilized, provided such material does not interfere with the articulating movement of the device.

The superior surface 11 of the superior member 10 is provided with a channel, depression, scallop or similar concave saddle-like structure 16 that is configured to receive the inferior edge of the superior spinous process 99. The inferior surface 22 of the inferior member 20 is also provided with a channel, depression, scallop or similar concave saddle-like structure 26 that is configured to receive the superior edge of the inferior spinous process 99. In one embodiment, the inferior surface 22 of the inferior member 20 is also provided on the anterior portion 14 with a pair of lateral flange members 27 that fit over the superior surface of the lamina on the inferior spinous process 99. These lateral flange members 27 serve to stabilize the device and prevent ventral migration into the spinal canal. In addition, stops, rails or even adjustable screws may be disposed on superior and inferior members 10 and 20 so as to restrain lateral movement relative to the spinous processes 99.

Anchoring means 30 may be provided to further secure the superior member 10 to the superior spinous process 99 or lamina and to secure the inferior member 20 to the inferior spinous process 99 or lamina. The anchoring means 30 may comprise any suitable means suitable for fixed attachment of objects to the spinous process 99 or vertebra, such as straps, mechanical fasteners or, as shown, tensioning cords 31 disposed through bores 32.

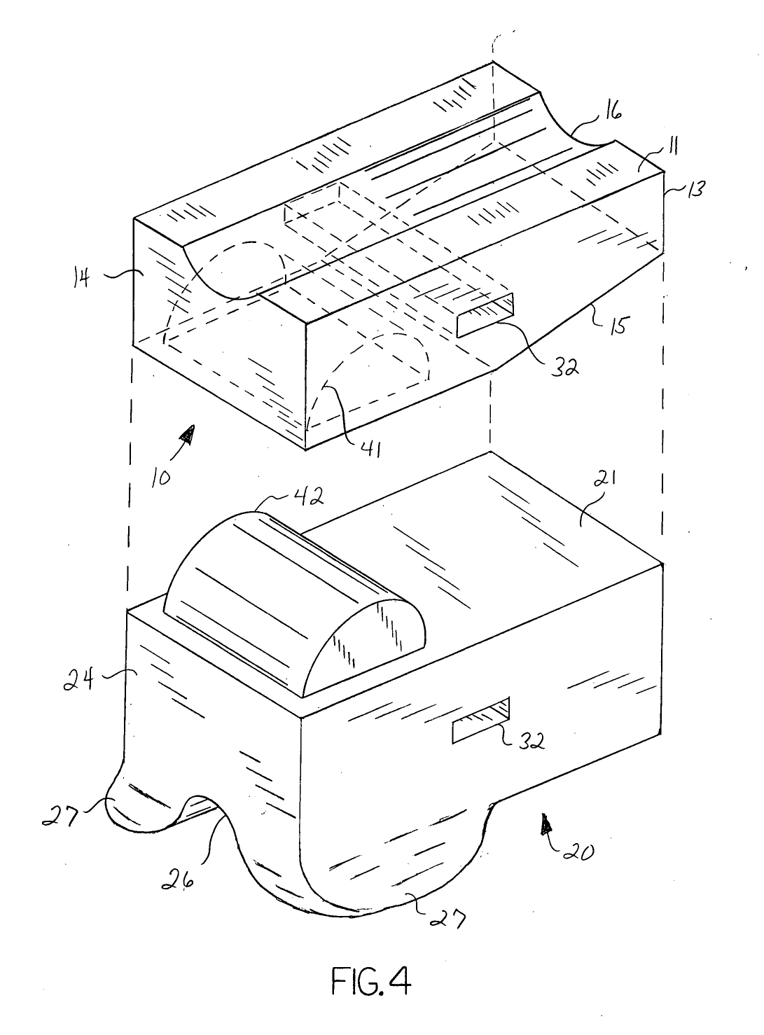

Articulating means 40 are provided such that the superior member 10 and the inferior member 20 may move relative to each other in flexion, extension, rotation, lateral bending and translation in any plane. As shown in FIGS. 1 and 2, articulating means 40 comprises a concave member 41 and a corresponding or mating convex member 42. Preferably, the concave member 41 and the convex member 42 are generally hemispherical, commonly referred to as a ball and socket configuration, such that inferior and superior members 10 and 20 may move in both a rotating and a pivoting manner, such that limited movement in all direction is allowed. Alternatively, other curvi-linear configurations other than hemispherical may be utilized in order to provide more or less relative movement. For example, a non-hemispherical curved surface may be provided that allows for relative lateral translation in one or more directions in addition to rotation and pivoting. For example, a semi-cylindrical configuration, as shown in FIG. 4, will allow only pivoting movement during flexion and extension of the spine, movement in the general horizontal or rotational direction being precluded.

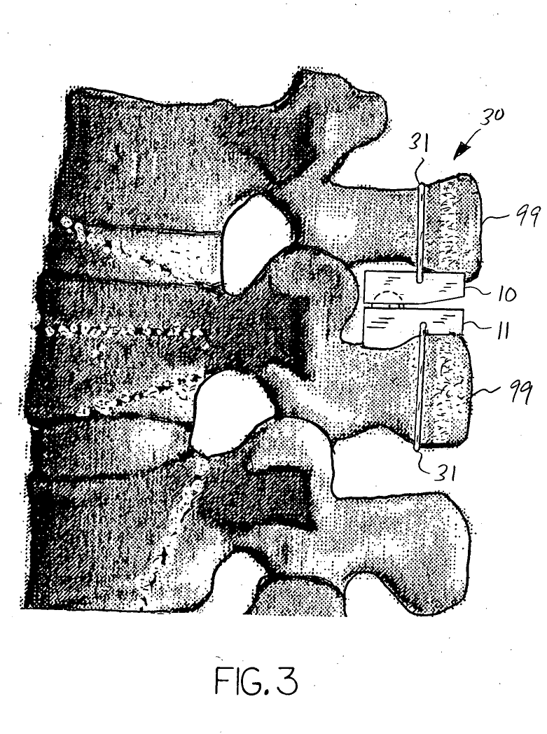

The articulating means 40 are disposed in the anterior portions 14 and 24 of the superior and inferior members 10 and 20, such that when the device is properly positioned in the spine the rotation and pivot point are posterior to the spinal canal and residing in the intervertebral foramina, as shown in FIG. 3.

In order to allow for relative movement in multiple directions, the depth of the convex or socket member 41 is less than that of the convex or projecting member 42, such that a gap is maintained between the superior surface 21 of the inferior member 20 and the inferior surface 12 of the superior member 10. Alternatively, the convex member 42 may be raised, mounted on a post, etc. such that the concave member 41 may pivot to a greater degree in multiple directions. It is to be understood, that the concave member 41 and convex member 42 may be reversed from the embodiment shown in the drawings, such that the concave surface 41 may be disposed on the inferior member 20 and the convex surface 42 disposed on the superior member 10.

To increase the range of pivot during extension of the spine, the posterior portion 13 of the inferior surface 12 of the superior member 10 is preferably provided with a beveled, slanted or curved surface 15 (the terms being used herein to depict functionally equivalent configurations) such that the gap between the two members 10 and 20 increases in dimension in the posterior direction. Preferably, a limited range of motion of approximately 8 degrees is desirable in flexion/extension. Likewise, it would also be possible to bevel, slant or curve the superior surface 21 of the inferior member 20 to define a beveled, slanted or curved surface 25 accomplish this, either in place of or in addition to the superior member 10. The anterior edges of the inferior surface 12 of the superior member 10 may also be beveled or radiused to allow for small movement in various planes during lateral movement flexion. To decrease the range of motion in certain directions, such as laterally for example, stop members such as rails, posts, shoulders, or even adjustable screws could be provided on one or both of the facing interior surfaces 12 and 21.

The device is inserted with the spinous processes 99 distracted, thereby placing the spine into segmental kyphosis. The combined anterior portions 14 and 24 prevent further flexion. The inferior surface 12 of the superior member 10 and the superior surface 21 of the inferior member 20 diverge in the sagittal plane and may converge in extension, with the amount of extension being limited by the angle of beveled surface 15. In this manner, the device distracts the spinous process 99, thus tensioning the ligamentum flavum and opening the neural foramen. The former may relieve an existing spinal stenosis and the latter decompresses the exiting nerve roots in the neural foramen, the net effect of which is to create segmental kyphosis. The center of rotation is behind the spinal canal and most likely unloads the disc in the neutral position and extension while minimizing the load in flexion.

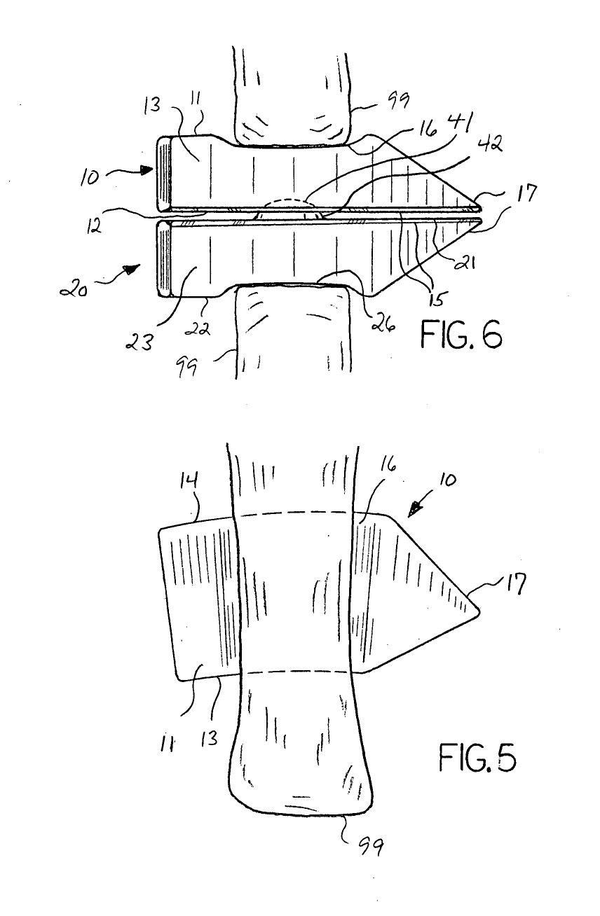

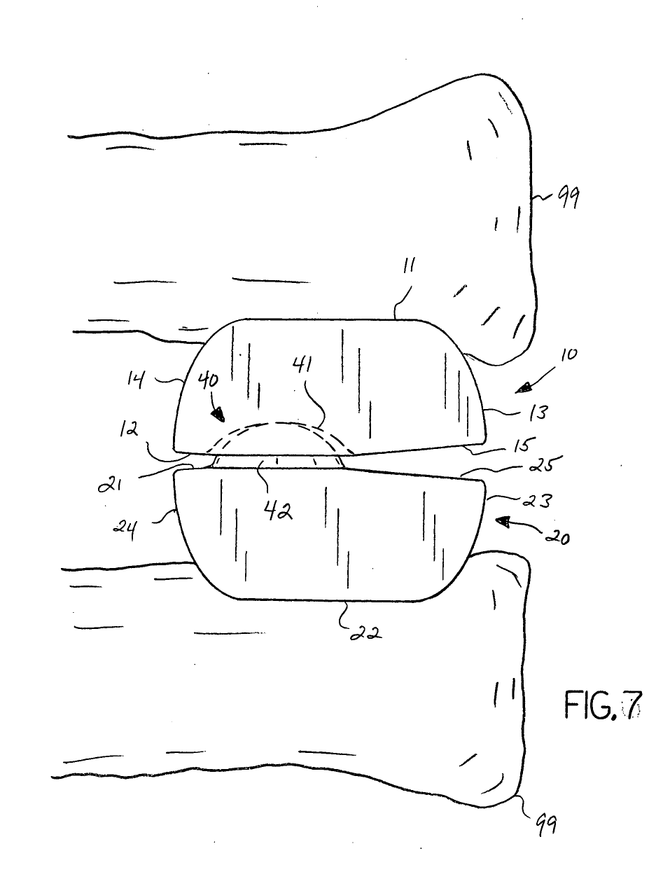

In an alternative embodiment depicted in FIGS. 5 through 7, the dynamic inter-spinous device is provided with a curved configuration in the lateral direction and a tapered or rounded leading edge or nose 17 to make insertion of the device between the spinous processes more easily accomplished. As shown in FIG. 7, the radius of curvature of the concave member 41 relative to the convex member 42 may vary, such that a relatively tight fit between the members 41 and 42 is not achieved and relative movement of the superior and anterior members 10 and 20 is not as tightly controlled.

It is contemplated that equivalents and substitutions for certain elements set forth above may be obvious to those skilled in the art, and therefore the true scope and definition of the invention is to be as set forth in the following claims.

I claim:

1. A dynamic inter-spinous device limiting and controlling the relative movement between adjacent spinous processes of the human body, said device comprising:
an inferior member and a superior member, wherein said inferior member abuts an inferior spinous process and said superior member abuts a superior spinous process; and
articulating means allowing relative movement of said inferior member and said superior member.

2. The device of claim 1, wherein said articulating means comprises a convex member received within a concave member.

3. The device of claim 2, wherein said articulating means are hemispherical.

4. The device of claim 2, wherein at least one of said articulating means is not hemispherical.

5. The device of claim 1, further comprising anchoring means securing said inferior member to said inferior spinous process and said superior member to said superior spinous process

6. The device of claim 5, wherein said anchoring means comprises one of a group consisting of straps, mechanical fasteners and tensioning cords.

7. The device of claim 2, wherein said superior member comprises a superior surface having a depression receiving said superior spinous process, and wherein said inferior member comprises a inferior surface having a depression receiving said inferior spinous process.

8. The device of claim 2, wherein said superior member comprises an inferior surface and said inferior member comprises a superior surface, and wherein at least one of said superior member inferior surface and said inferior member superior surface is beveled to increase the distance therebetween.

9. The device of claim 1, further comprising a tapered leading edge whereby insertion of the device between said adjacent spinous processes is more easily accomplished.

10. The device of claim 1, wherein said device is curved in the lateral direction.

11. The device of claim 1, wherein said articulating means allows relative movement of said inferior member and said superior member in flexion, extension, rotation, lateral bending and translation in any plane.

12. A dynamic inter-spinous device limiting and controlling the relative movement between adjacent spinous processes of the human body, said device comprising:
an inferior member and a superior member, wherein said inferior member abuts an inferior spinous process and said superior member abuts a superior spinous process; and
articulating means allowing relative movement of said inferior member and said superior member in flexion, extension, rotation, lateral bending and translation in any plane, said articulating means comprising in combination a concave member and a convex member, wherein said convex member is received within said concave member, and wherein said concave member is disposed on one of said inferior or superior members and said convex member is disposed on the other of said inferior or superior members.

13. The device of claim 12, wherein said convex member and said concave member are hemispherical.

14. The device of claim 12, wherein said convex member and said concave member are not hemispherical.

15. The device of claim 12, wherein said superior member comprises a superior surface having a depression, and wherein said inferior member comprises an inferior surface having a depression.

16. The device of claim 12, wherein said superior member comprises an inferior surface and said inferior member comprises a superior surface, and wherein at least one of said superior member inferior surface and said inferior member superior surface is slanted to increase the distance therebetween.

17. The device of claim 16, wherein said slanted surface increases the distance between said superior member inferior surface and said inferior member superior surface in the posterior direction.

18. The device of claim 12, further comprising a tapered leading edge.

19. The device of claim 12 wherein said device is curved in the lateral direction.

20. The device of claim 12, further comprising anchoring means securing said inferior member to said inferior spinous process and said superior member to said superior spinous process, wherein said anchoring means comprises one of a group consisting of straps, mechanical fasteners and tensioning cords.
